(19) United States
(12) Patent Application Publication
(10) Pub. No.: US 2021/0346114 A1
(43) Pub. Date: Nov. 11, 2021

(54) APPARATUS, SYSTEMS AND METHODS FOR NEGATIVE PRESSURE FACE SHIELDING

(71) Applicant: University of Iowa Research Foundation, Iowa City, IA (US)

(72) Inventors: Henry Hoffman, MD, Iowa City, IA (US); Jarrett Walsh, MD, Coralville, IA (US)

(21) Appl. No.: 17/317,615

(22) Filed: May 11, 2021

Related U.S. Application Data
(60) Provisional application No. 63/023,019, filed on May 11, 2020.

(51) Int. Cl. A61B 90/00; A61B 90/40

(52) U.S. Cl. CPC A61B 90/05; A61B 2090/502; A61B 90/40

(57) ABSTRACT

The disclosed apparatus, systems and methods relate to devices, systems and methods relating to a negative pressure face shield comprising a housing defining a lumen, at least one anterior access port defined within the housing, and a suction port defined within the housing.

### CROSS-REFERENCE TO RELATED APPLICATION(S)

This application claims the benefit under 35 U.S.C. § 119(e) to U.S. Provisional Application 63/023,019, filed May 11, 2020, and entitled “Apparatus, Systems and Methods for Negative Pressure Face Shielding,” which is hereby incorporated by reference in its entirety for all purposes.

### TECHNICAL FIELD

The disclosed technology relates generally to devices, systems and methods designed to enhance the safety of aerosol generating health care procedures by limiting health care provider exposure to aerosols.

### BACKGROUND

The Corona Virus Disease 2019 (COVID-19) pandemic has radically altered the approach to medical care and has impacted the management of disorders of the upper aerodigestive tract. The risk of viral spread to health care workers through the aerosol generating procedure of trans-nasal laryngoscopy is significant.

It is recognized that high energy surgical devices such as laser and electrocautery produce plumes that contain cardiotoxic and carcinogenic aerosols as well as viable viral contaminants. Efforts to control spread of these substances include recommendations from the Centers for Disease Control and Prevention (CDC) to limit dispersion through use of smoke evacuators containing a suction unit.

Concern about risk of viral transmission associated with aerosol generating procedures has been amplified during the COVID pandemic resulting in intensified efforts to limit dissemination of surgically produced aerosols.

Thus, there is a need in the art for a novel shield designed to establish a negative pressure microenvironment about the face of the patient for use during trans-nasal and transoral procedures.

### BRIEF SUMMARY

The disclosure relates to apparatus, systems and methods for prevention of airborne transmission of infection via a negative pressure face shield. Various implementations of the shield utilize a form factor that maximizes the ability to disinfect it between uses, while others relate to a single-use device.

Example 1 relates to a negative pressure face shield comprising, a housing defining a lumen; at least one anterior access port defined within the housing; and a suction port defined within the housing.

Example 2 relates to the negative pressure face shield of Example 1, further comprising flange at an inferior portion of the housing, the flange configured for attachment to a stand.

Example 3 relates to the negative pressure face shield of Example 1, further comprising a sealant disposed around the at least one anterior access port.

Example 4 relates to the negative pressure face shield of Example 3, wherein the sealant comprises one or more of tape, adhesive, gel, oil, wax, epoxy, or jelly.

Example 5 relates to the negative pressure face shield of Example 1, wherein the housing has a depth of at least about 3.5 inches.

Example 6 relates to the negative pressure face shield of Example 1, wherein the anterior access port is disposed at a lower midline of the housing.

Example 7 relates to the negative pressure face shield of Example 1, wherein the suction port is disposed on a side of the housing.

Example 8 relates to a face shield comprising: a housing comprising a face, a superior portion, an inferior portion, and at least first side portion and a second side portion. The face shield of Example 8 further includes at least one access port defined within the face, at least one suction port defined within the first side portion, and a hood engaged with at least the superior portion of the housing, wherein the at least one suction port is configured to be engaged with a filtration system, and wherein the housing and hood define a lumen where a hermetic environment can be created.

Example 9 relates to the face shield of Example 8, further comprising at least one flange disposed on the inferior portion.

Example 10 relates to the face shield of Example 8, further comprising a sealant disposed around the at least one access port to prevent fluid or pressure transfer across the face via the access port.

Example 11 relates to the face shield of Example 10, wherein the sealant is removable such that the access port may be used for insertion of tools into the hermetic environment.

Example 12 relates to the face shield of Example 8, wherein the hood comprises a fabric.

Example 13 relates to the face shield of Example 8, wherein the housing is sterilizable.

Example 14 relates to the face shield of Example 8, wherein the housing is acrylic.

Example 15 relates to a system for minimizing aerosol spread comprising a negative pressure face shield comprising, a housing defining a lumen, at least one anterior access port defined within the housing, at least one suction port defined within the housing, and a hood engaged with the housing for encircling a head of a patient. The system of Example 15 also includes a filtration system in communication with the at least one suction port via tubing, wherein a negative pressure environment is created within the lumen.

Example 16 relates to the system of Example 15, further comprising a tie for securing the hood to the patient.

Example 17 relates to the system of Example 15, wherein one or more tools may be inserted into the negative pressure environment via the at least one anterior access port.

Example 18 relates to the system of Example 15, wherein the hood is engaged with the housing via one or more of a strap, Velcro, adhesive, glue, grommets, buttons, tape, and male/female connectors.

Example 19 relates to the system of Example 15, wherein the hood is comprised of a surgical fabric.

Example 20 relates to the system of Example 15, wherein the housing is sterilizable.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1A is a front view of the protective system, according to one implementation.

FIG. 1B is a perspective view of the protective system, according to one implementation.

FIG. 1C is a close-up view of the protective system, according to one implementation.

FIG. 2A is a close-up view of the protective system in place near an exam chair, according to one implementation.

FIG. 2B is a perspective view of the protective system of FIG. 2A, according to one implementation.

FIG. 3A is a side view of the protective system on an exam chair with a hood, according to one implementation.

FIG. 3B is a perspective view of the protective system of FIG. 3A, according to one implementation.

FIG. 3C is a front view of the protective system of FIG. 3A, according to one implementation.

FIG. 4A is a front perspective view of a NPFS, according to one implementation.

FIG. 4B is a rear perspective view of the NPFS of FIG. 4A, according to one implementation.

FIG. 5A is a front perspective view of a NPFS, according to one implementation.

FIG. 5B is a rear perspective view of the NPFS of FIG. 5B, according to one implementation.

FIG. 6 is a side view of a NPFS, according to one implementation.

FIG. 7A is a perspective view of a NPFS, according to one implementation.

FIG. 7B is a front view of the NPFS of FIG. 7A, according to one implementation.

FIG. 7C is a side view of the NPFS of FIG. 7C, according to one implementation.

FIG. 8A is a perspective view of a NPFS, according to one implementation.

FIG. 8B is a side view of the NPFS of FIG. 8A, according to one implementation.

FIG. 9A is a side view of a NPFS, according to one implementation.

FIG. 9B is a side of the NPFS of FIG. 9A in use, according to one implementation.

FIG. 10A is a front view of a NPFS, according to one implementation.

FIG. 10B is a front schematic view of a NPFS, according to one implementation.

FIG. 100 is a close-up view of a flap arrangement, according to one implementation.

FIG. 11 is a front view of the protective system with micronanometer, according to one implementation.

FIGS. 12A-D shows the placement of the protective system on a patient for use, according to one implementation.

FIG. 13A is a front view of the protective system in place on a patient, according to one implementation.

FIG. 13B is a side view of the protective system in place on a patient of FIG. 13A, according to one implementation.

FIG. 13C is a side view of the protective system in place on a patient of FIG. 13A, according to one implementation.

FIG. 13D is a rear view of the protective system in place on a patient of FIG. 13A, according to one implementation.

FIG. 14A is a front view of the protective system in place on a patient, according to one implementation.

FIG. 14B is a perspective view of the protective system in place on a patient of FIG. 14A, according to one implementation.

FIG. 14C is a perspective view of the protective system in place on a patient of FIG. 14A, according to one implementation.

FIG. 14D is a perspective view of the protective system in place on a patient of FIG. 14A, according to one implementation.

FIG. 15 is a side view of the protective system in use, according to one implementation.

FIG. 16 is a side view of the protective system in use, according to one implementation.

FIG. 17 is a side view of the protective system in use, according to one implementation.

FIG. 18 is a side view of the protective system in use, according to one implementation.

FIG. 19A is a close-up view of the protective system in use, according to one implementation.

FIG. 19B is a close-up view of the protective system in use, according to one implementation.

FIG. 20 is a graph showing oxygen saturation during procedures where the protective system was used.

FIG. 21 is a graph showing tolerability of the NPFS and protective system during procedures.

FIG. 22 is a graph showing oxygen saturation during procedures where the protective system was used.

FIG. 23 is a graph showing tolerability of the NPFS and protective system during procedures.

FIG. 24 is a graph showing tolerability of the NPFS and protective system during procedures.

### DETAILED DESCRIPTION

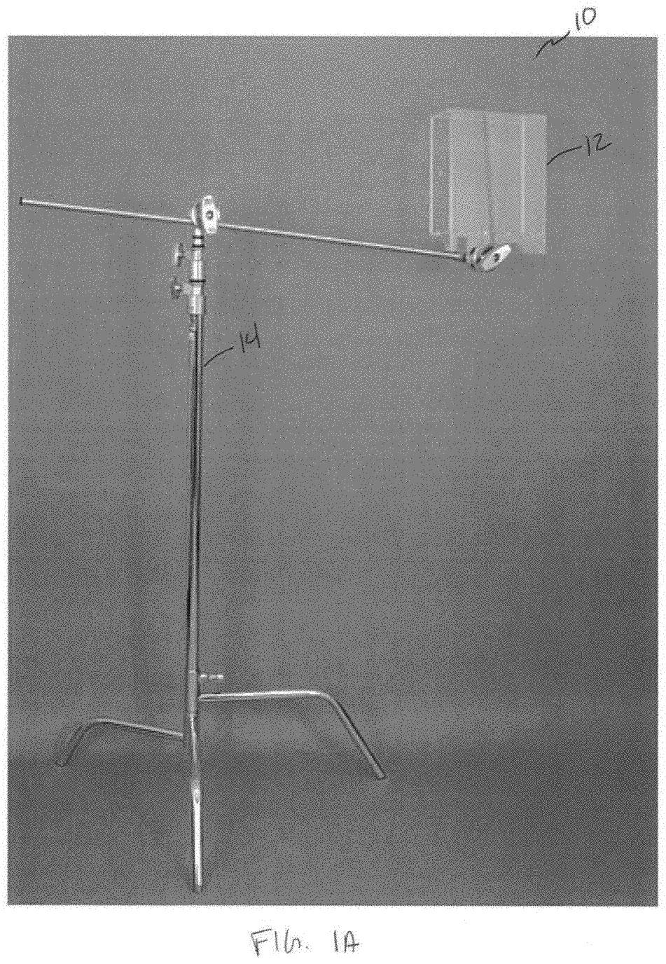

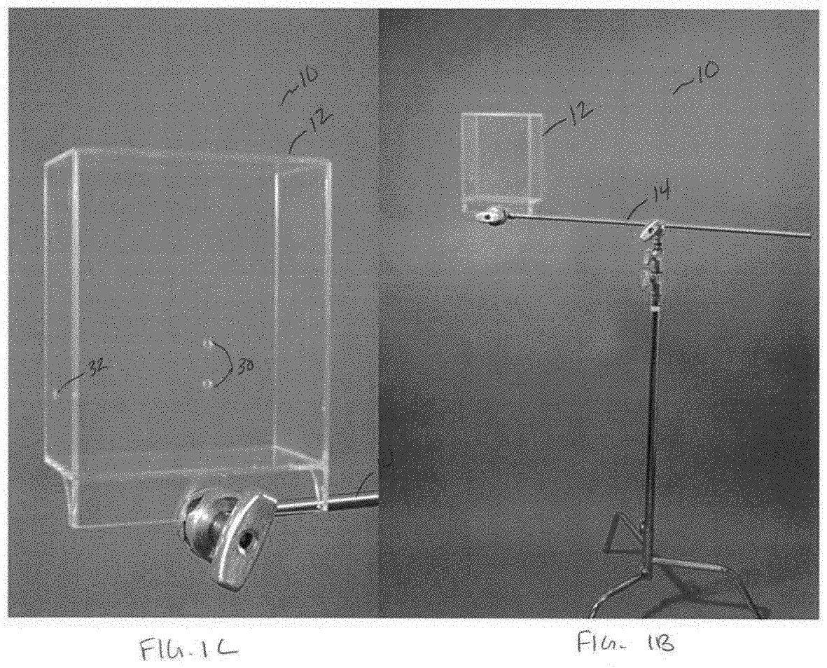

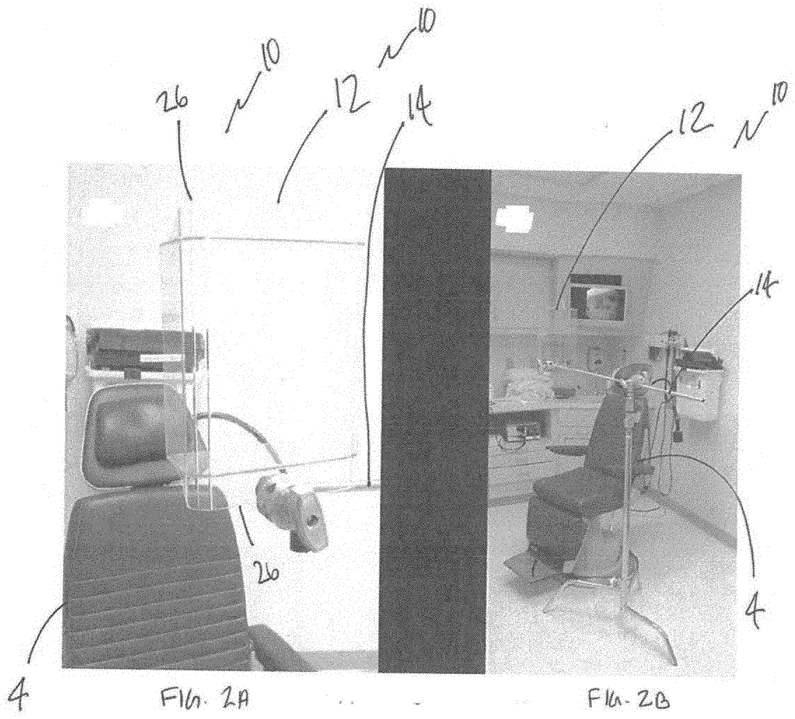

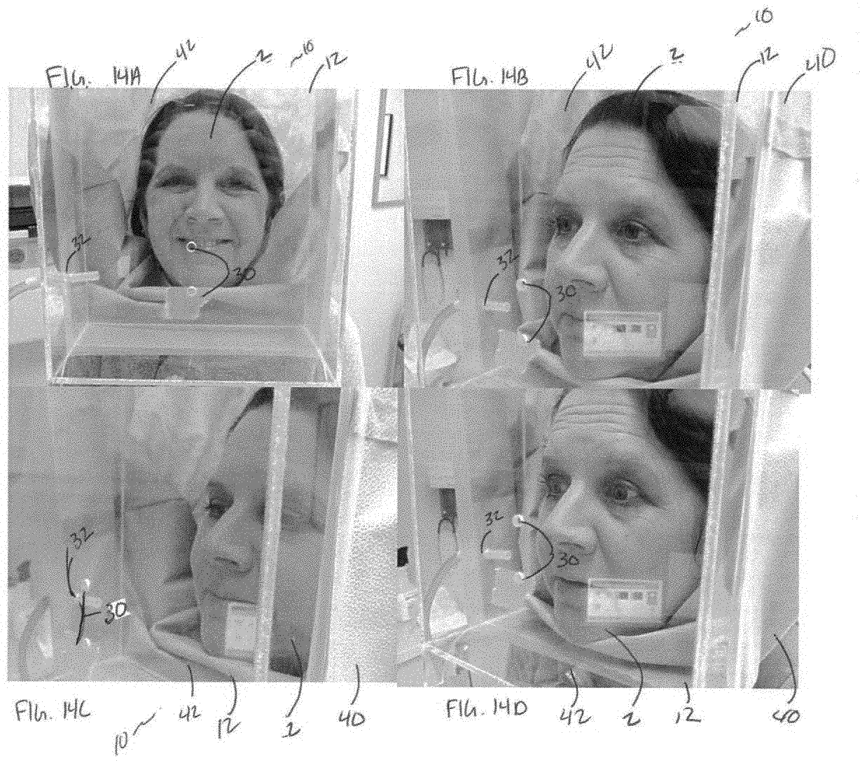

Discussed herein are various devices, systems and methods that relate to a protective system 10 including a negative pressure face shield (NPFS) 12, shown variously in FIGS. 1A-19B. Various implementations of the NPFS 12 and protective system 10 allow for increased safety during aerosol generating medical procedures. The protective system 10 is configured to create a negative pressure environment around a patients head via the NPFS 12 and other components such that aerosols generated by the patient during a procedure are directed towards a filter or otherwise away from the medical practitioner. As such, infectious aerosols are filtered rather than entering the ambient air and creating a health and/or safety hazard of the medical practitioner or other individuals in the vicinity of the patient.

Turning to the figures in greater detail, in various implementations like that shown in FIG. 1A-3C, the protective system 10 comprises an NPFS 12 that can be affixed to a stand 14. In various implementations, the NPFS 12 and/or stand 14 are single use or replaceable, as would be readily appreciated by those of skill in the art. A variety of stands 14 can be utilized, as would be readily appreciated by those of skill in the art. In certain implementations, the NPFS 12 are reusable and sterilizable.

As shown for example in FIGS. 2A-2B the NPFS 12 can be positioned near an exam chair 4 for use in a medical procedure. For placement, the NPFS 12 may be releasably attached to the stand 14 via a flange 26. In certain implementations, the flange 26 is at the bottom edge of the NPFS 12 in other implementations, the NPFS 12 include more than one flange 26, shown for example in FIG. 2A for further flexibility in system 10 arrangement.

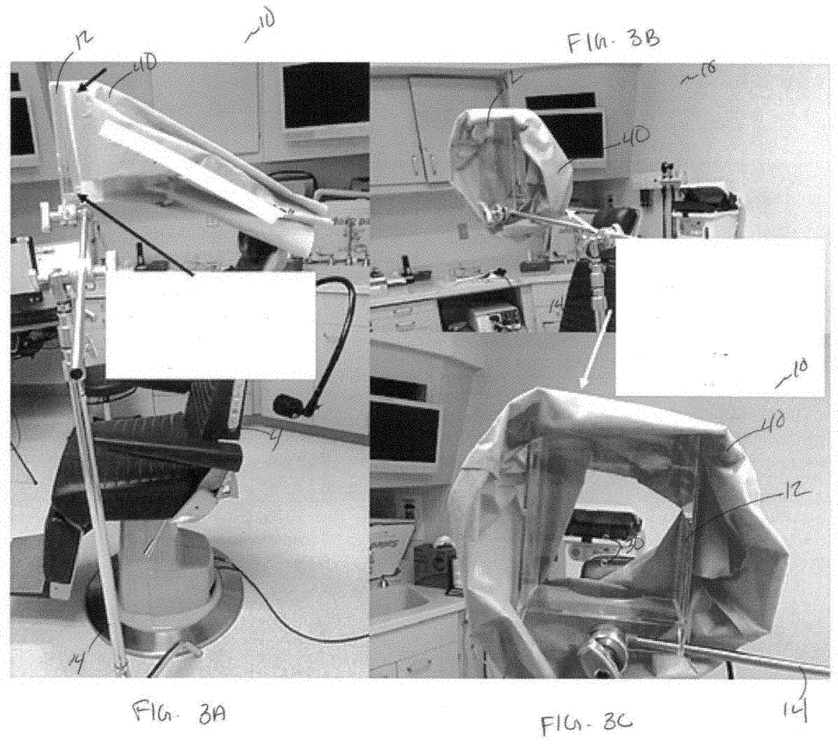

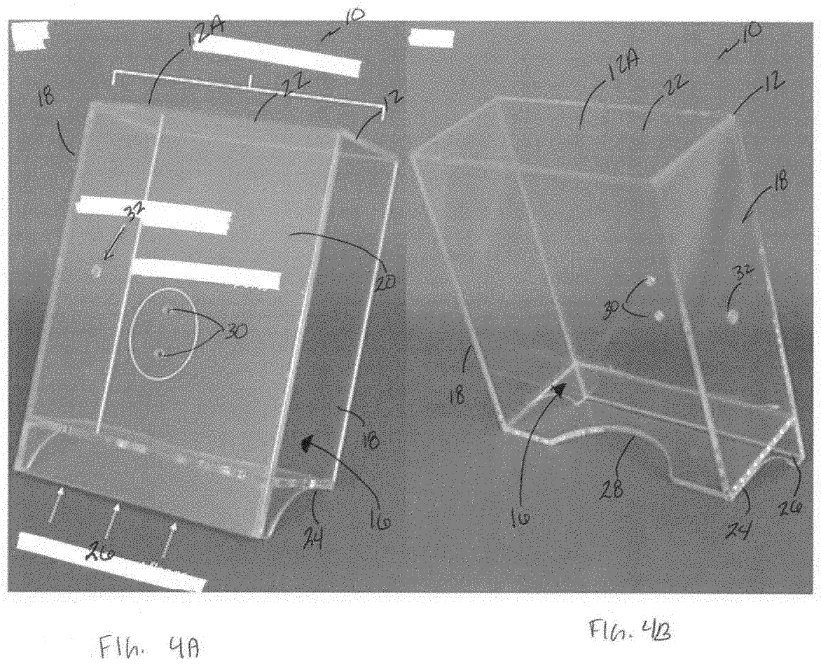

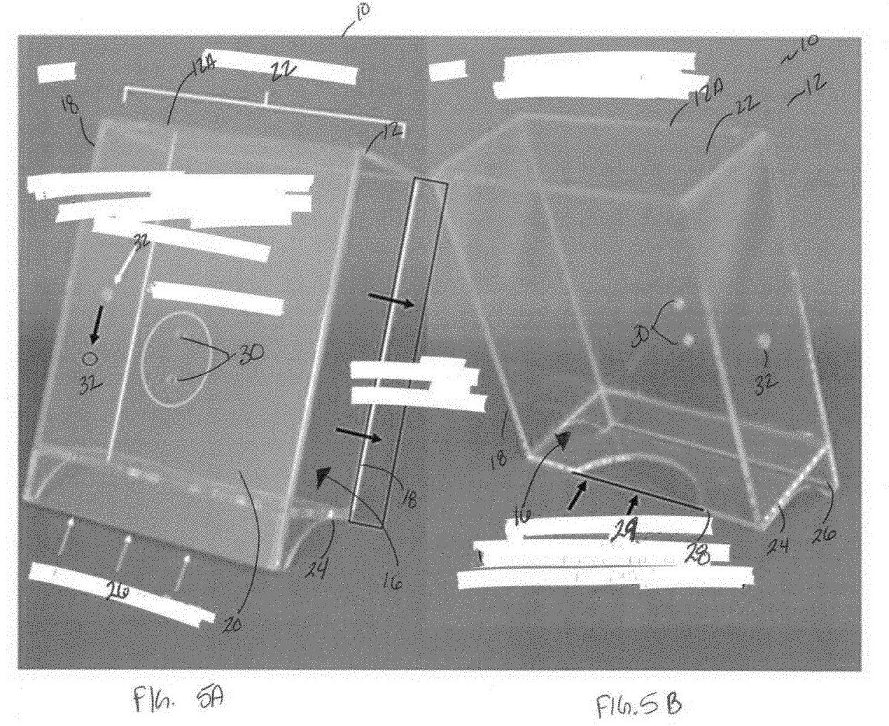

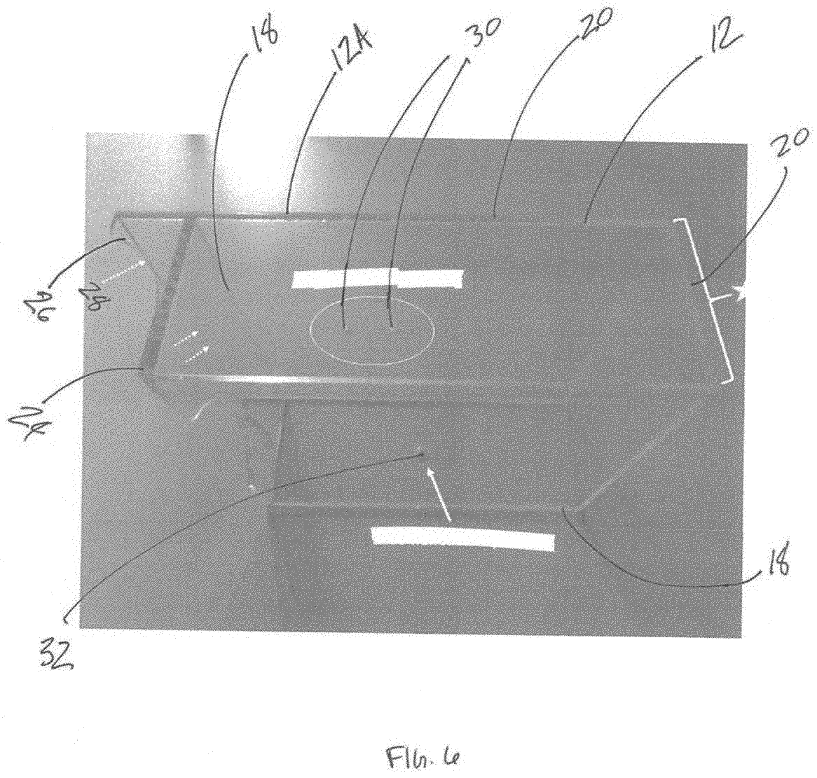

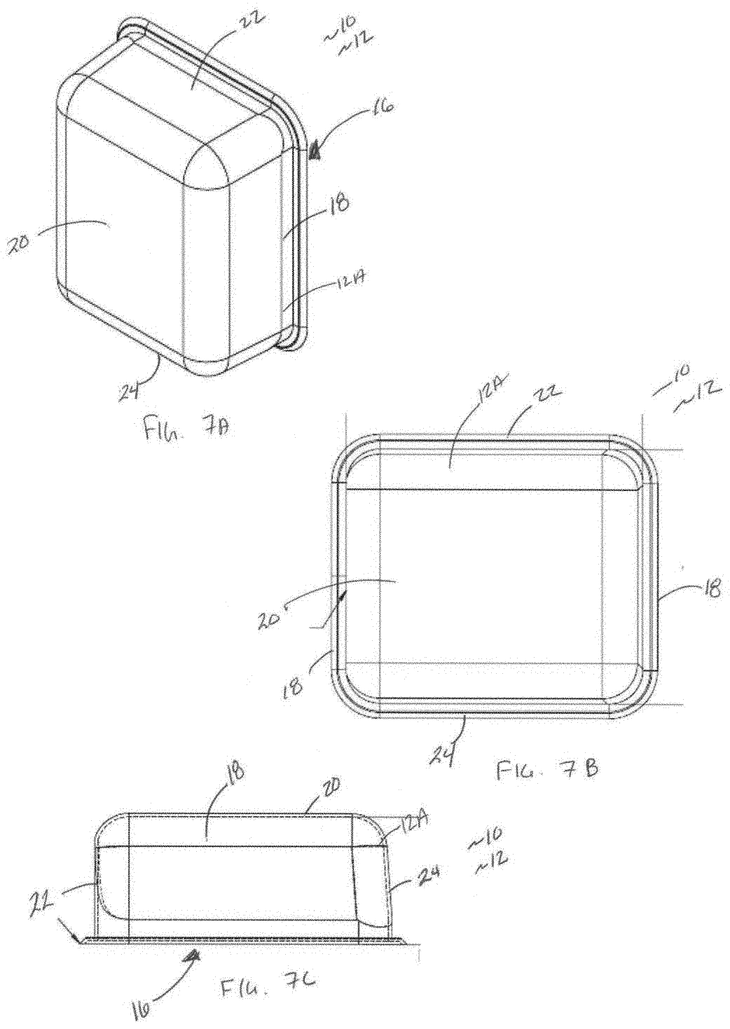

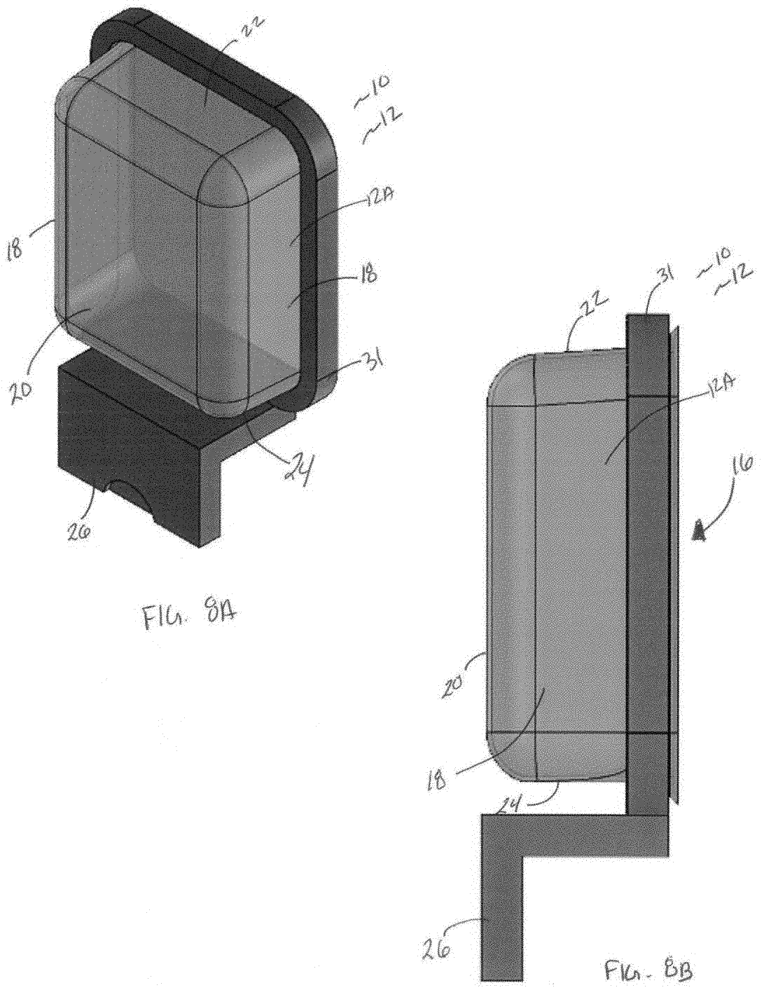

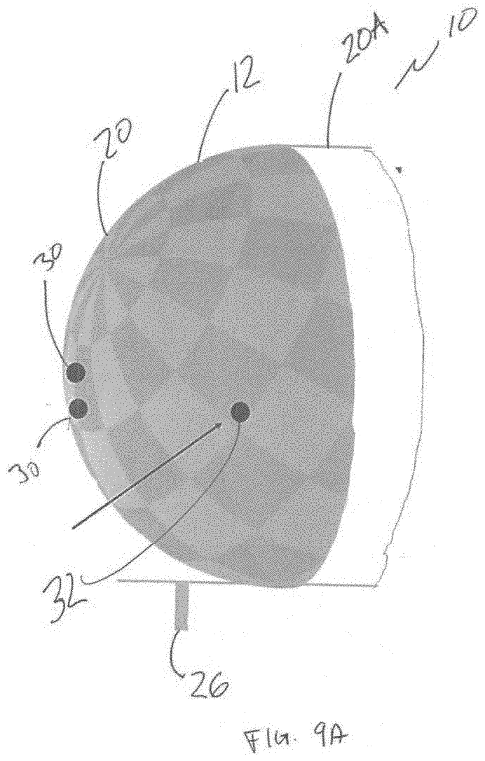

The various implementations shown in FIG. 3A-3C show the protective system 10 with the optional hood 40, as will be discussed further below. In these and other implementations, the hood 40 is arranged to be draped over the head of a patient to create the negative pressure environment.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed systems and methods, the subject has been diagnosed with a need for treatment of one or more otolaryngology or dental procedures prior to the treatment step.

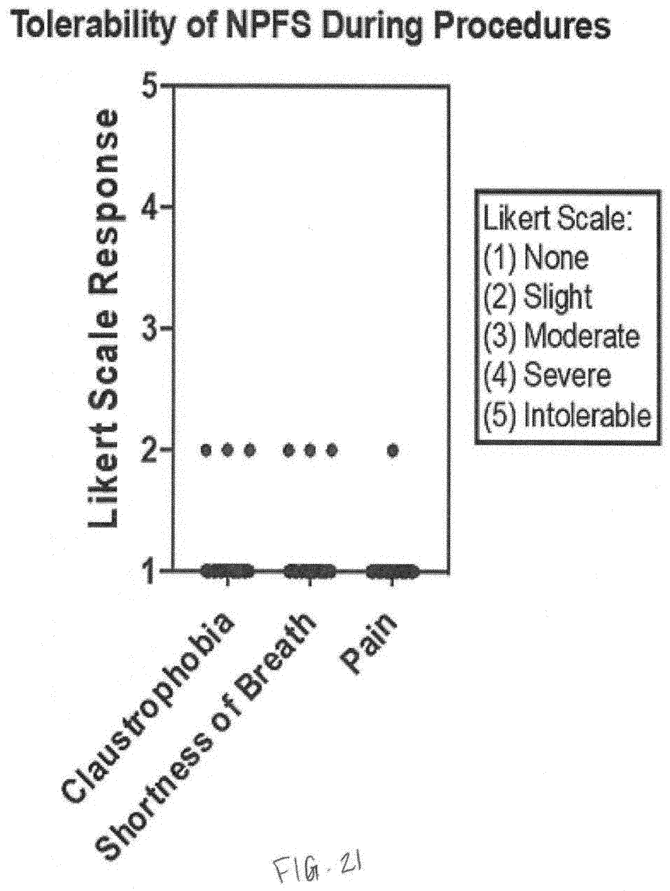

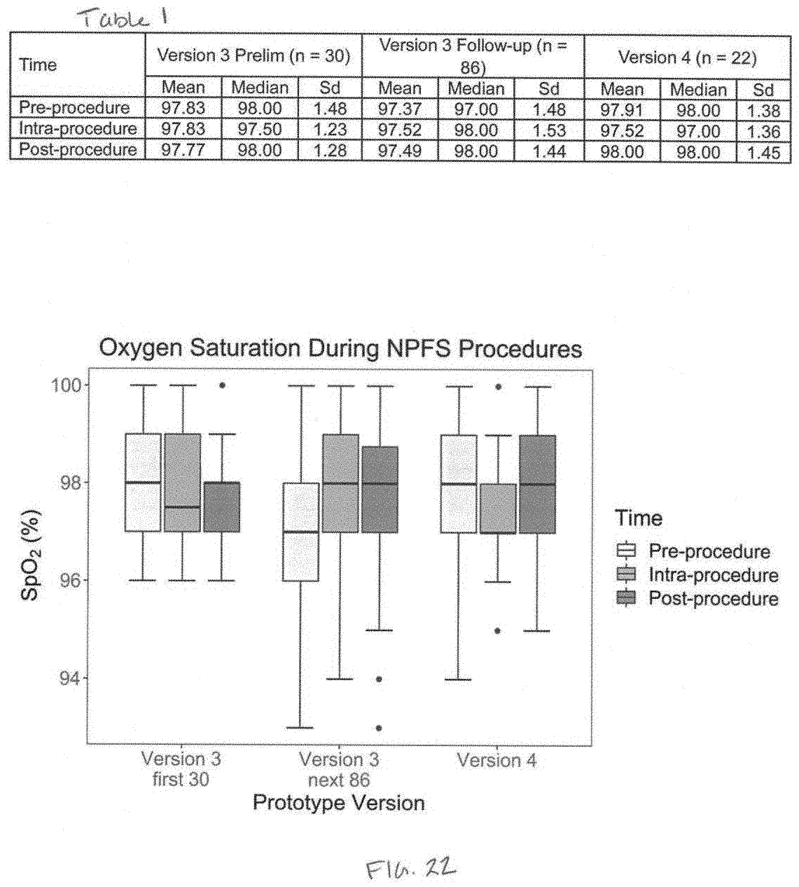

In one embodiment, as shown in FIGS. 2A-8B, the negative pressure face shield (NPFS) 12 has a housing 12A that is a substantially transparent device 12 defining a lumen 16 sized to accommodate the face of a patient 2. In various implementations, the device 12 may be made of acrylic, or other suitable material as would be recognized by those of skill in the art. The NPFS 12 housing 12A according to certain implementations can also be comprised of any of a large variety of transparent or semi-transparent rigid, non-porous materials capable of sterilization. While the implementations of FIGS. 1A-8B depict a polygonal NPFS 12, it is readily appreciated that in alternate implementations the NPFS 12 comprises an alternate shape, such as the rounded configurations shown in FIGS. 9A-9B, where an optional posterior projection 20A is provided to extend the distance between the patient 2 and the face of the NPFS 20/access ports 30. It is appreciated that many alternate forms and configurations are of course possible.

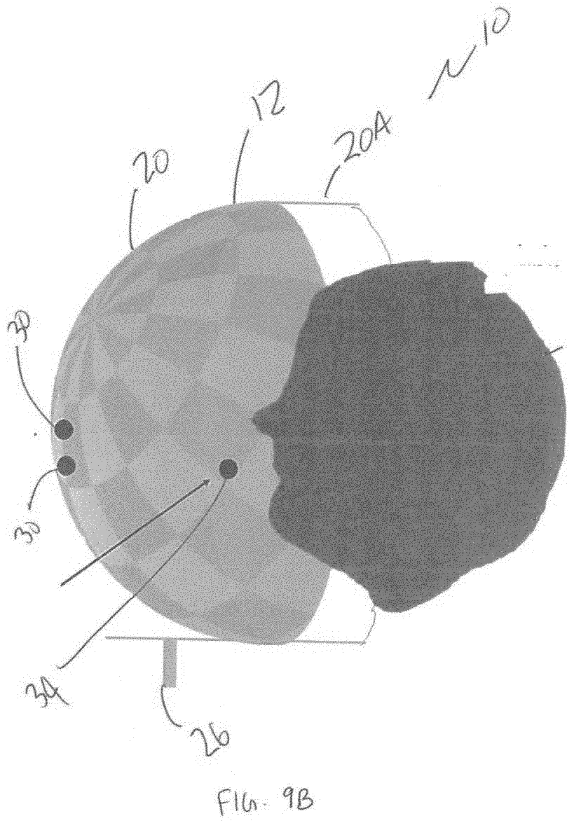

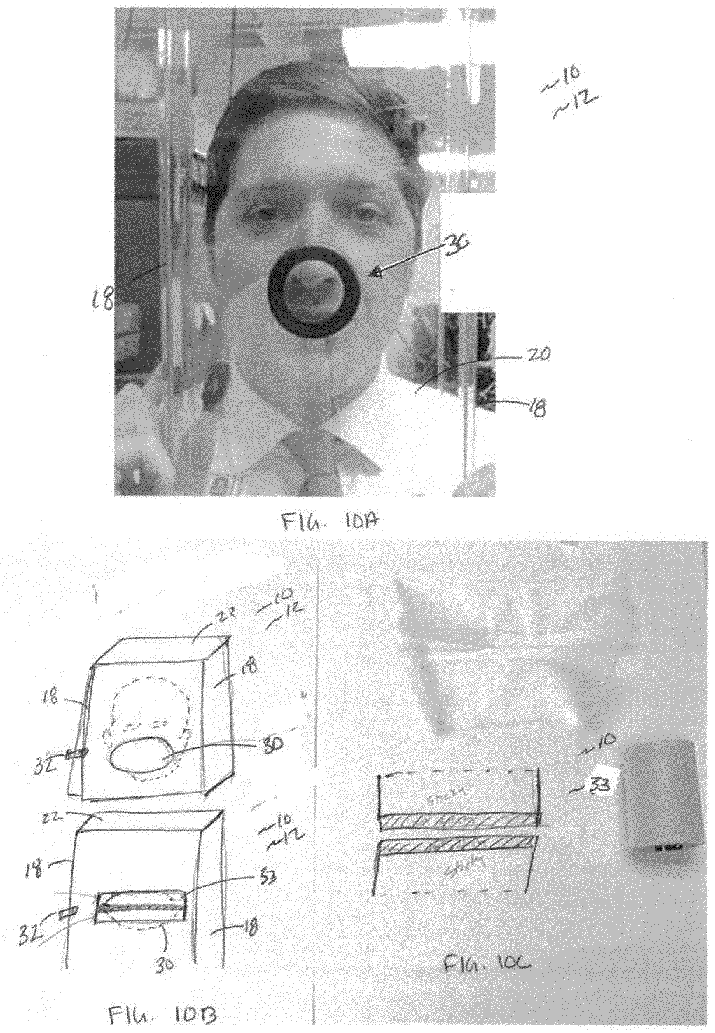

Continuing with the implementation of FIGS. 4A-4B, in the NPFS has sides 18, a face 20, as well as a top 22 and bottom 24, thereby defining a height, depth and width of the lumen 16, as would be readily appreciated. It is further appreciated that in certain implementations, the shape of the NPFS 12 allows for the quick and easy sterilization of the NPFS 12 between procedures such that it may be re-used. That is, the size and planar nature of the various sides 18, 20, 22, 24 allows for complete treatment with sterilizer and quick drying, in these implementations. Various implementations are structured to ensure the absence of irregular surfaces that could potentially trap infectious material inaccessible to cleaning with antimicrobial wipes and sprays. Alternate implementations are single use.

As is further shown in the implementation of FIGS. 4A-4B, an optional stabilization flange 26 is also disposed from the top 22 (as shown in FIG. 2A) and/or the bottom 24. In various implementations, the flange 26 is used engage a clamp (again shown best in FIG. 2A) on the stand 14 for positioning in front of the patient, as would be readily appreciated and/or to aid in securing a hood 40, as described further below. In the implementations of FIGS. 4A-B, the bottom 24 further defines a neck opening 28 sized to accommodate the neck of a patient when the patient's face is in the lumen 16, as would be readily appreciated.

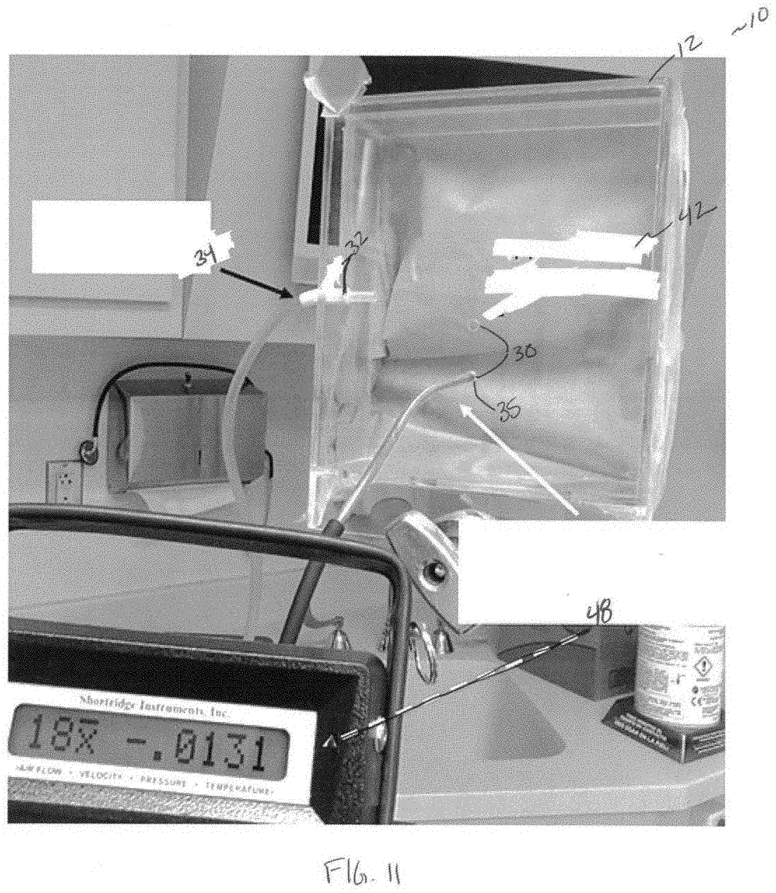

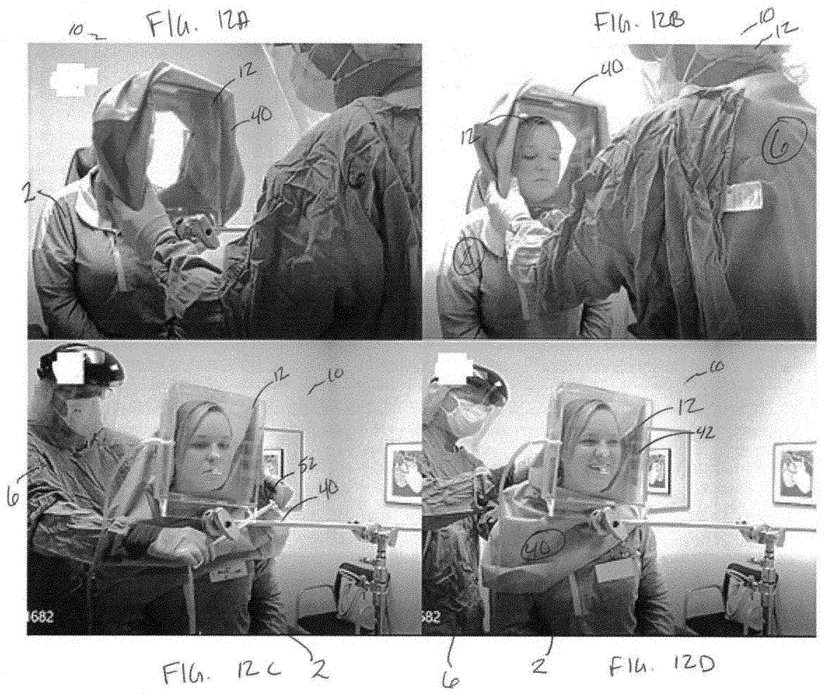

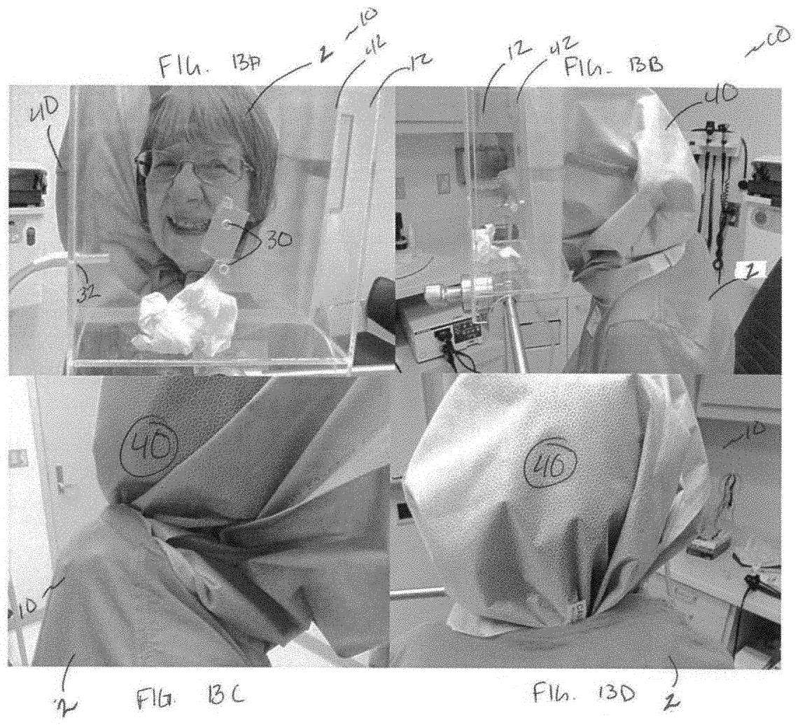

As shown in FIG. 5A the sides 18 of the NPFS 12 may be extended to create a deeper lumen 16, where the patient may be spaced further apart from the face 20 while maintaining the negative pressure environment. As shown in FIG. 5B, the NPFS may not include the neck opening 28 (shown in FIG. 4B) rather the bottom 24 may be straight across. In certain implementations, the bottom may act as a chin rest.

Continuing with FIGS. 2A-6, these and other implementations of the NPFS 12 comprise one or more facial, or anterior access ports 30 defined in the face 20 sized for the insertion of various surgical or procedural tools used in medical procedures. For example, in various implementations the anterior access ports 30 can be sized to accommodate tools in a variety of procedures performed on the head, neck throat, mouth, eyes, teeth, ears and/or upper body of a subject or patient, as well as various dental tools, as would be readily appreciated by those of skill in the art. It is readily appreciated that the one or more access ports can be provided at a variety of locations on the face 20 so as to facilitate a number of specific procedures or uses, such that the ports 30 may be defined relatively more caudally or rostrally, as well as toward either side 18 of the NPFS 12.

As is also shown in FIGS. 2A-6, the NPFS 12 according to these implementations also features a suction port 32 that can be defined in a side 18. It is readily appreciated that in alternate implementations the suction port 32 can be defined in other locations such as the top 22 or bottom 24 of the NPFS 12. As shown in FIGS. 4A-B the suction port 32 may be substantially at the mid-point of the side 18. Shown in FIG. 5A, it may be advantageous in certain circumstances for the suction port 32 to be lower on the side. Of course alternative placements and orientations are possible.

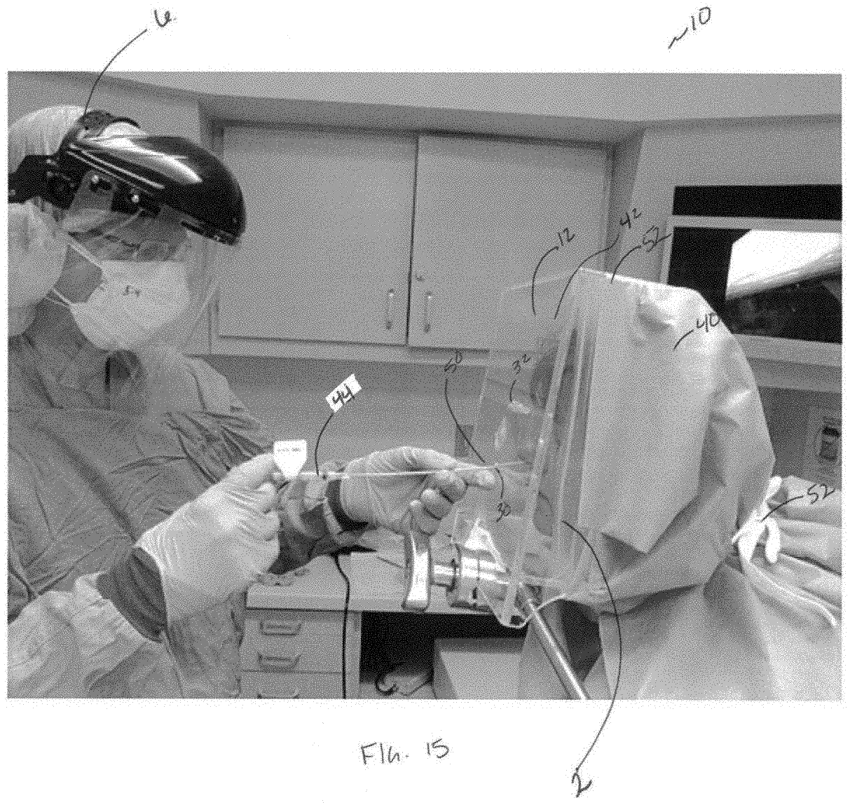

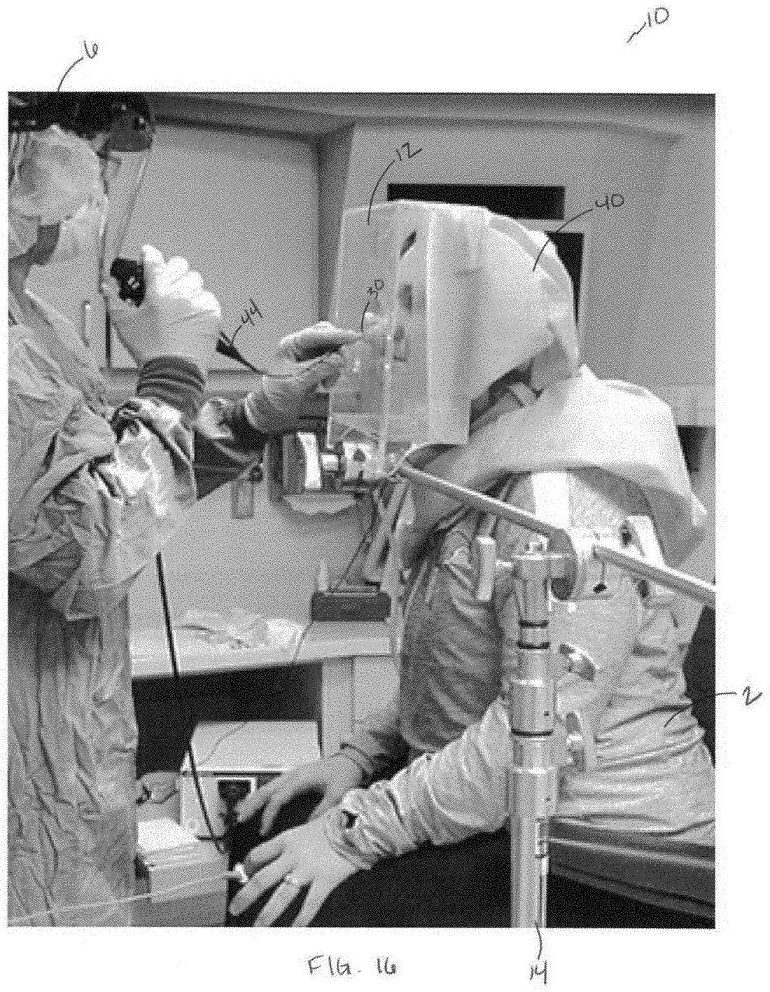

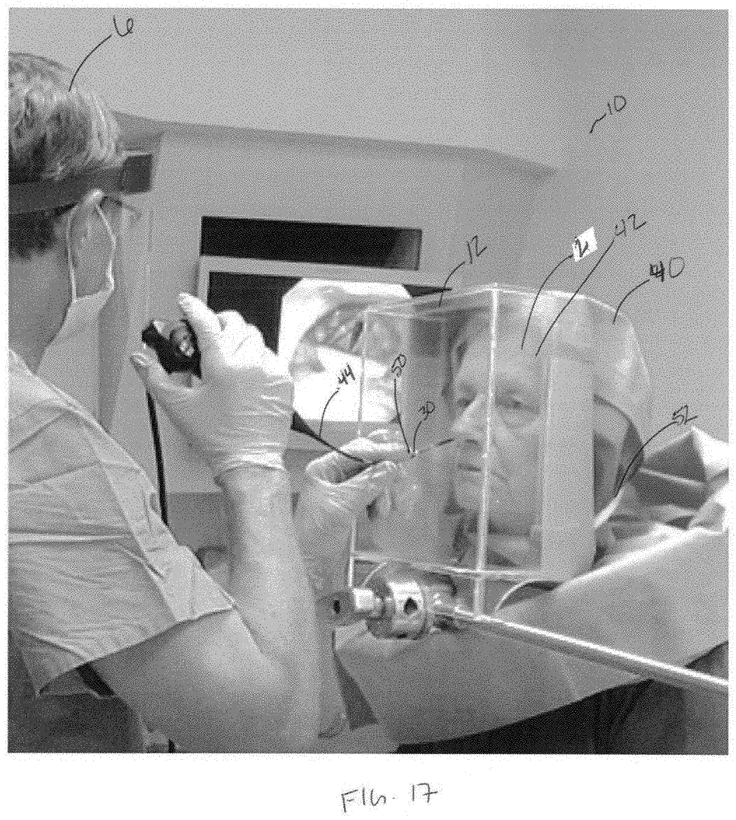

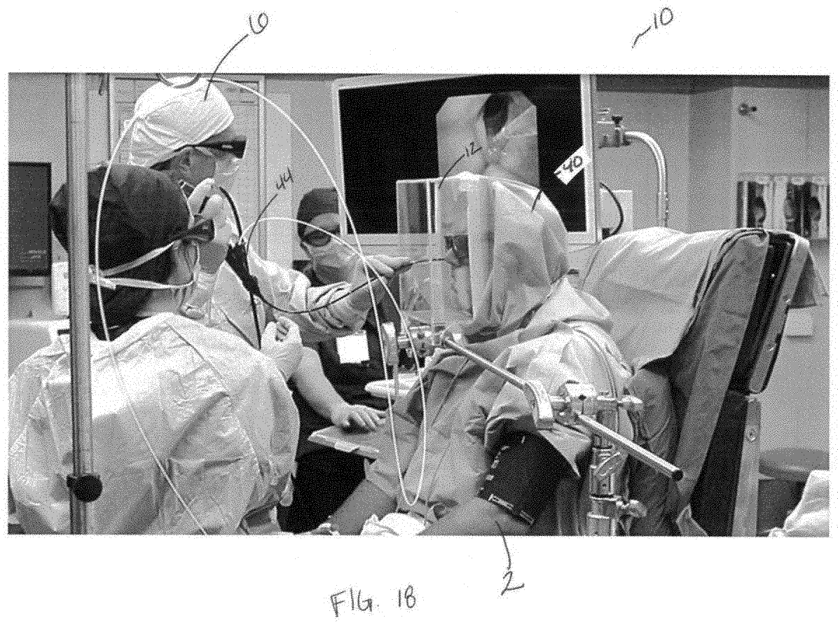

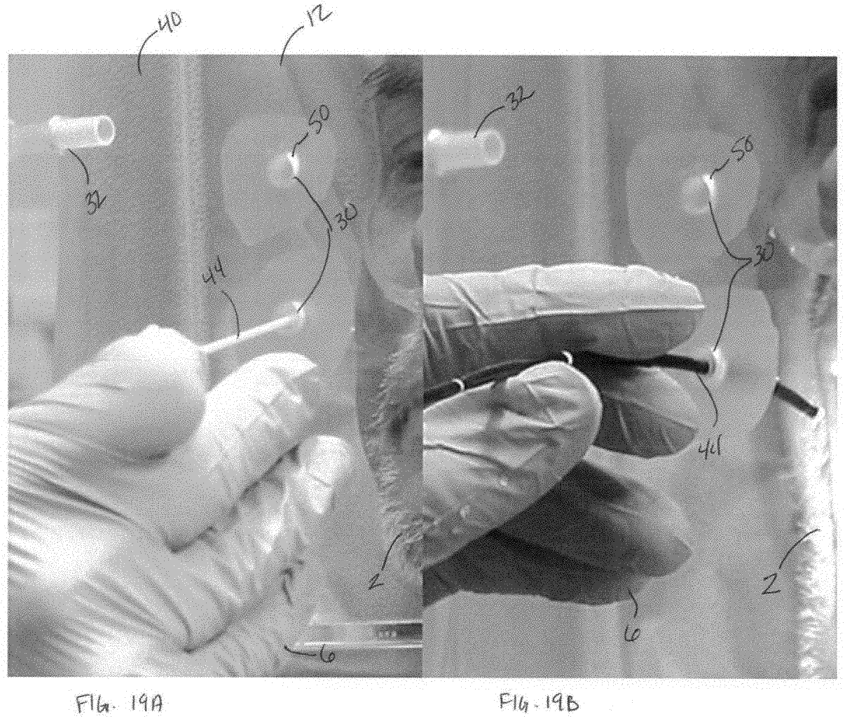

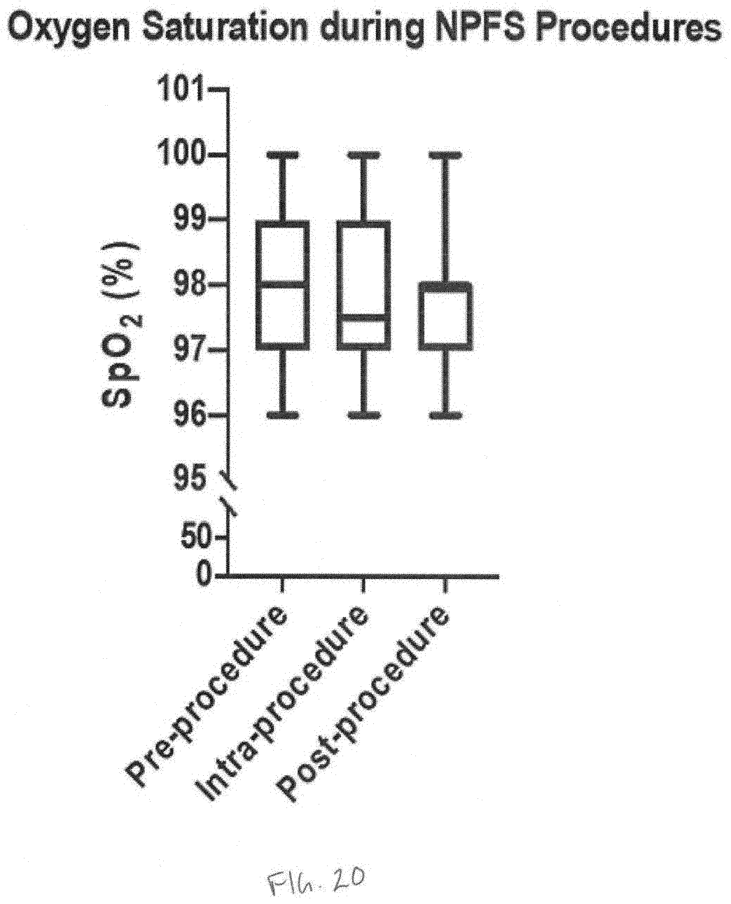

FIGS. 7A-8B show alternative implementations of the NPFS 12. The NPFS 12 in FIGS. 7A-C includes rounded corners, which may allow for enhanced cleaning and sterilization between uses. FIGS. 8A-B show an implementation of the system 10 where the NPFS 12 is insertable into a frame 31. In various implementations, the flange 26 is apart of the frame 31 which can thereby be attached to a stand 14.

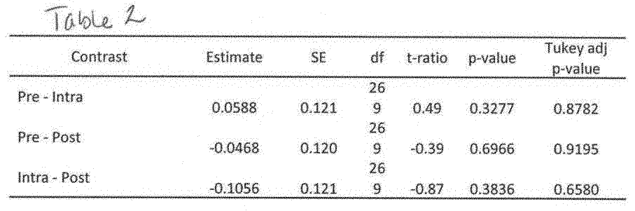

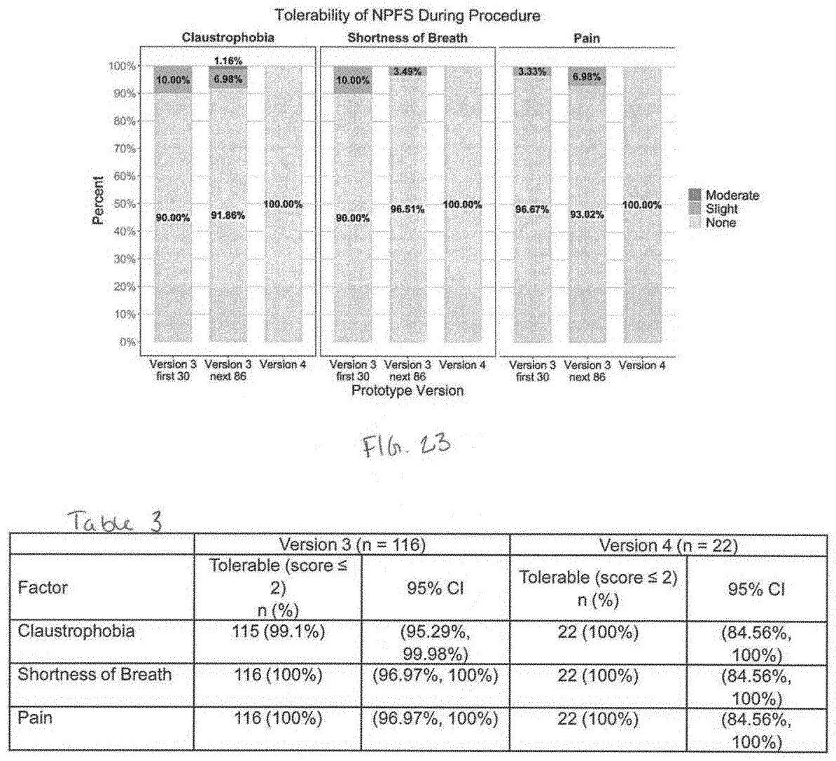

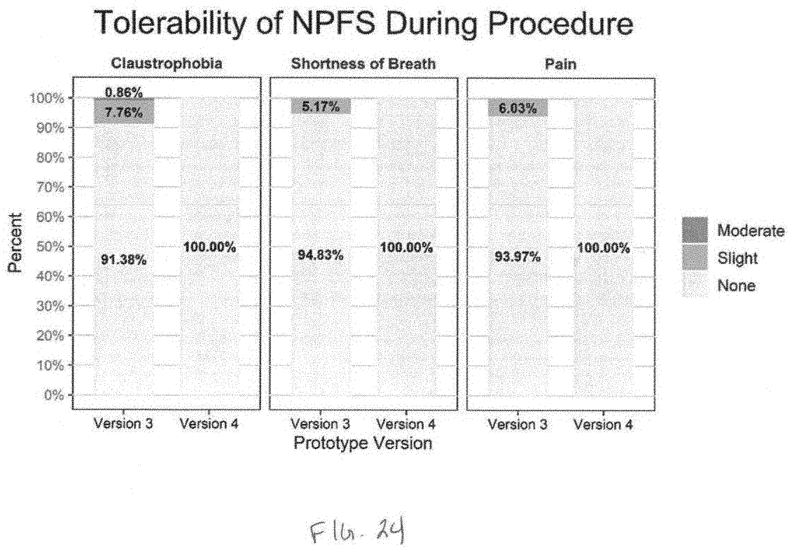

FIGS. 10A-C depict a further alternative implementation of the NPFS 12 having a relatively larger access port 30 when compared to the ports 30 shown in FIGS. 1A-9B. In certain implementations, the larger port 30 may be used for insertion of larger tools and devices. In these and other implementations, the access port 30 may include an optional flap 33. The flap 33 may be configured to allow for insertion of a tool while maintaining the negative pressure environment in the lumen 16. For example, the flap 33 may deform to fit the shape of the tool, as would be appreciated. The flap 33 may be affixed to the face 20 via any known mechanism such as adhesive.

As shown in the examplary system 10 depicted in FIG. 11, the NPFS 12 suction port 32 is sized or otherwise constructed and arranged to be in fluidic or hermetic communication with a vacuum tube 34, such a suction tube 34 typically provided in procedure or operating rooms from a central system or those that can be provided on a cart for certain specialized procedures.

In use according to various implementations, and as shown in FIGS. 12A-19B, the NPFS is oriented over the face of the patient 2, who is in turn optionally in an exam chair (as shown best in FIGS. 2A-B at 4). A hood 40 is secured around the head of the patient 2 to create a hermetic microenvironment 42 where the application of suction or vacuum to the microenvironment 42 creates a negative pressure microenvironment (again shown at 42). FIGS. 12A-D show an exemplary step by step process of arranging the system 10 and creating the hermetic microenvironment 42 around a patient's head. FIGS. 13A-D show various view of the system 10 and NPFS 12 after a hermetic microenvironment 42 is established. As will be discussed in more detail below, the hood 40 may be secured with a tie or strap 52 (shown in FIGS. 12C-D) or by otherwise securing the hood 40 such as by tucking the ends of the hood 40 into the patient's shirt/top/clothing (shown in FIGS. 13B-D). Further views of the system 10 with a hermetic microenvironment are shown in FIGS. 14A-D.

It is appreciated that in exemplary embodiments, the system 10 provides a sustained or continuous negative pressure microenvironment 42 throughout the duration of the procedure, reducing the spread of any aerosolized contagions between the patient 2 and physician 6 and reducing the spread of disease such as COVID-19 and the like.

It is appreciated that in various implementations, the vacuum introduced by the vacuum tube 34 to the suction port 32 and therefore microenvironment 42 can be in the range of about 1 to about 300 mmHg, or from about 1 to about 530 mmHg or more while the physician 6 is introducing various tools 44 into the microenvironment 42 for use in the patient 2 procedure, shown for example in FIGS. 15-19B. In various implementations, the vacuum tube 34 may be in operational communication with a control unit 48 (shown for example in FIG. 11) configured to operate and/or monitor the applied suction such as an electronic micromanometer like an Airdata® Multimeter ADM-870C from Shortridge Instruments, Inc. In various implementations, the suctioned air is filtered through a filtration system such as a HEPA system such that air drawn from the microenvironment 42 over the pressure differential can be safely removed from the general air around the patient 2 and physician 6.

Continuing with the use of the system 10, in the implementations of FIGS. 15-19B the physician is introducing on or more tools 44 through the access port(s) 30 in the NPFS 12. It is appreciated that these tools 46 can include scopes, cameras, lights, and the like. Certain non-limiting examples include flexible laryngoscopes, laser tools, bronchoscopes, intranasal cryotherapy devices, intranasal balloon catheters, eustachian tube balloon catheters, and various tools for injections, retrobulbar injections, intranasal injections, control of hemostasis, application of drugs, biopsies, visualization, resection and the like. Those of skill in the art would readily appreciate the application not only for otolaryngology, dental and ophthalmologic procedures, but for a wide range of medical procedures that involve close contact between care providers and subjects or patients, particularly but not limited to those including the use of tools in and around the head and body.

As shown in the implementations of the system 10 shown FIGS. 19A-B, the NPFS 12 can have a plurality of anterior access ports 30 that may or may not be in use according to certain specific implementations. In these and other implementations, a sealant 50 is provided to prevent fluid and pressure transfer over any unused port(s) 30, as would be readily understood. Further, in various implementations the sealant 50 can serve to prevent leakage through an in-use port by sealing any space between the walls of the port 30 and the tool 44 body, as would also be readily understood. In various implementations, the sealant 50 can be tape or other adhesive, or any of a number of gels, oils, waxes, epoxies, jellies or the like. In various implementations, the sealant is removable and/or replaceable such that the sealant may be in place when needed to seal a port 30 but can be removed such that the port 30 can be accessed.

Returning to the hood 40 shown variable in FIGS. 3A-C and 12A-18, in these implementations the hood 40 according to various implementations can be a light, breathable or semi-breathable cloth, such as surgical fabric similar to Halyard® H100 Surgical Sterilization Wraps. Alternate implementations may use H500 Wraps or other surgical wraps understood in the art of sufficient size, weight and porosity to facilitate the creation of the microenvironment 42 capable of sustaining a negative pressure while minimizing patient discomfort and claustrophobia. It is appreciated that the microenvironment 42 may be assessed for pressure via a control unit 48 in fluidic communication with the microenvironment 42 via a testing tube 35 inserted via an access port 30, as is shown in FIG. 11 and discussed below in the Examples.

As is also shown in FIGS. 3A-C and 15, in certain implementations, the hood 40 is secured to the NPFS 12 via a strap 52 or other securement such that the hood 40 and NPFS 12 are separable for cleaning and/or disposal of one or more of the components. While a strap 52 is shown in FIGS. 3A-C and 15, alternate implementations utilize Velcro®, adhesive, glue and/or bonding agent, grommets, buttons tape and/or mated male/female connections or other approaches known and understood in the art for tightly securing a fabric to a solid such as acrylic. In alternate implementations, the hood 40 is affixed to the NPFS 12 for single use implementations of the system 10.

### EXAMPLES

### Example 1

Objective. Develop methods and materials to limit droplet and microdroplet spread in the performance of trans-nasal aerosolizing generating procedures

### Study Design/Materials and Methods

Prototype development and prospective patient series addressing feasibility using flexible trans-nasal laryngoscopes inserted into the access ports of the NPFS.

NPFS. Testing of multiple prototypes led use in patients of the negative pressure face shield (NPFS) a transparent 9″×10.5″ rectangular acrylic device with a depth of 3.5″ and an inferior stabilization flange used to engage a clamp on a stand for positioning in front of the patient. The stand is designed as a heavily weighted camera stand with a clamp permitting engagement of the flange (Matthews Hollywood Centeruy 40″ S Stand For Grip Arm Kit Adorama Inc. New York, N.Y.).

Prototype face shields were adapted and tested initially on human volunteers and then on 10 successive patients in the course of creating a suction-clearing negative-pressure microenvironment surrounding the face providing access to the nose and mouth. Evaluation included pressure measurements within prototypes followed by prospective patient evaluation clinically treated with the device including assessment through questionnaires and monitoring oximetry.

Design characteristics included ensuring the absence of irregular surfaces that could potentially trap infectious material and be inaccessible to cleaning with antimicrobial wipes and sprays. The three openings in the NPFS were to be smooth and sealed to prevent trapping of particles. These openings included two separate ¾″ access ports (holes) in the lower midline of the face shield and a ¾″ suction port on the mid-lateral surface for placement of suction.

Pressure. Experiments included initial testing with standard wall suction (maximum regulated suction of 320 mm Hg±20) (Vacutron® Suction Regulators by Chemetron Inc). Additional testing of prototypes included trial of the portable Neptune® 3 Waste Management System 120 Vac Rover which includes a High Efficiency Particular Air Filter (HEPA) and is capable of generating continuous suction in a range from 50 to 520 mm HG (Neptune 3 IFU 0703-001-700.pdf). Initial testing in human volunteers included assessment of fluctuations in the negative pressure generated within the shield during inspiration and ventilation. Additional pressure testing was done employing a single versus double layer of the sterilization wrap as well as comparison of use of the wall suction on maximum suction (300+/−20 mm Hg) versus the Neptune suction (520 mm Hg maximum) suction with a HEPA filter.

All patient related experiments were performed in a standard examination room designated as having 6 air changes per hour (ACH) employing the Vacutron® suction device attached to centralized suction created for the hospital environment that is filtered at the intake and exhaust sides with a 95% efficiency filter. The sterile disposable suction tubing with connector (0.6 mm×3.7 mm non-conductive suction tubing (12 ft length) CarinalHealth® Waukegan Ill.) was used consistently.

Hood. The design of the Halyard® “grade H100” sterilization wrap as a cylinder was exploited to secure it with tape circumferentially around the NPFS and then drape over the patient's head to create a closed environment by closing the lower aspect of the drape with a loose tie around the patient with a 3-4 foot length of umbilical ties. The H100 sterilization wrap is ‘a disposable infection control product’ made of water-repellant breathable “low lint SMS fabric” (SMS=spunbond meltblown spunbond) as is also used in production of face masks and lab jackets.

The prepared NPFS with sterilization wrap applied is rotated into position with the patient leaning slightly forward and raising or lowering the exam chair to a level optimal for the exam with attention to one of the two anterior access ports chosen for use. The port not in use was taped shut. Twill tape (½ inch umbilical tape) is placed around the neck to improve the seal to ensure negative pressure within the device.

An electronic micromanometer (Airdata® Multimeter ADM-870C Shortridge Instruments, Inc.) was used to measure pressure at the anterior port in the face shield to test the impact of variables.

Testing included assessment of the amount of negative pressure generated within the face shield placing the testing probe within one of the ports and with the H100 sterilization wrap in place around the shield. Testing was done both with the second anterior port opened and with it closed with tape.

Measurements were made through a series of modifications including use of towels and blankets positioned around the posterior aspect of the shield to provide a simple occlusion method with readily available materials to maximize the negative pressure generated. Ongoing assessments of patient comfort identified no discomfort or difficulty in breathing.

Procedure. Assessment of airflow and negative pressure was made with suction placed through 0.75 cm opening in the lateral aspect of the shield with the pressure/density multimeter occluding the opening in the anterior shield.

Assessments included measurements during inspiration, expiration, and at rest employing suction at different levels.

A mock-up of trans-nasal laryngoscopy was then performed. Before performing the transnasal laryngoscopy each of the 10 patients received 1 cc of a mixture of 4% lidocaine with 1% phenylephrine delivered to the nostril through an anterior access port by syringe spray employing the MADgic® Laryngo-tracheal mucosal atomization device (Teleflex Medical, Inc Morrisville, N.C.).

Duration. The duration of the procedure which was measured from the initial placement of shield and wrap to its removal and included the nasal spray, waiting for it to work, and the performance of the transnasal laryngoscopy ranged from 2 minutes 45 seconds to 6 minutes 5 seconds. The longest procedure included interaction with a speech pathologist to elicit additional vocalization maneuvers.

Pulse oximetry. Pulse oximetry was performed on all subjects before, during, and after the procedure. A questionnaire was applied immediately after the procedure to address comfort, degree of claustrophobia and shortness of breath. Comments from the patient about the experience were solicited and recorded.

Pulse oximetry done prior to placement of the shield, during the procedure with the shield and drape in place, and following completion of the procedure showed no drop in oxygenation during the process. One patient with compromised pulmonary function was maintained on treatment with continuous 3 liters of oxygen per minute by nasal cannula with oxygenation remaining at 100% throughout the process.

### Results

Negative pressure measurements at the anterior opening of the NPFS with a single layer of the grade H100 sterilization wrap identified a mean of negative 0.013 wi (inches of water) obtained from 18 samples over a 3 minute interval. This finding was reproduced without change both with the second port open and with it closed with tape.

Variables tested included measurement at rest, during inspiration, and during expiration. Additional variables included assessment of sequential modifications to the size and shape of the shield as well as methods to occlude the region around the patients face (combinations of towels, blankets, and positioning). Depending on the cycle of respiration, employing a single wrap and wall suction, the negative pressure varied between 0.002 (expiration) to 0.020 (inspiration) wi. More consistent negative pressure at higher levels was achieved with a double layer of fabric and higher negative pressure was achievable (530 mm Hg) with the Neptune suction.

Continuous pulse oximetry monitored the subjects oxygenation with frequent inquiries into the comfort of the process. No statistical changes in pulse oximetry were noted from baseline observations.

A negative pressure face shield (NPFS) was developed as a transparent easily sterilized acrylic shield with side suction port and two anterior access ports. This NPFS was successfully used to perform trans-nasal laryngoscopy within an isolated negative pressure microenvironment created about the face. Patient tolerance was high as determined by pulse oximetry (all saturations maintained above 96%) and a questionnaire immediately after the procedure identifying no shortness of breath (10/10), no claustrophobia (9/10) and no pain (9/10). Patient comments ranged from “I think this was a very good idea with Covid Virus” to “I thought it was absolutely ridiculous”.

Conclusion. Success in performing diagnostic laryngoscopy with a device designed to limit dispersion of aerosols employing a face shield to create a negative pressure microenvironment around a patient's face. Further development is anticipated to extend its application beyond diagnostics to include trans-nasal laryngeal laser and biopsy procedures. Additional modifications and testing are under way to permit performance of intranasal and intraoral procedures (including dentistry) performed in a similar protected environment.

Prototype testing. Negative pressure measurements at the anterior ports of the NPFS with a single layer of the grade H100 sterilization wrap hood identified a mean of negative 0.013 wi (inches of water) obtained from 18 samples over 3 minutes. This finding was reproduced without change both with the second port open and with it closed with tape.

### Example 2

Prototypes of a negative pressure face shield (NPFS) were tested then used clinically to create a suction-clearing negative pressure microenvironment with controlled access to the nose and mouth. Air pressure measurements within prototypes were followed by prospective evaluation of 30 consecutive patients treated with the device assessed through questionnaires and monitoring oximetry.

The NPFS is a transparent acrylic barrier with two anterior instrumentation ports and a side port to which continuous suction is applied. Testing of multiple prototypes led to development of the NPFS made of 0.2″ (5 mm) thick acrylic. This NPFS is a transparent 9″×10.5″ rectangular device with a depth of 3.5″ and an inferior stabilization flange used to engage a clamp on a stand for positioning in front of the patient. A heavily weighted camera stand employs a clamp engage the flange (Matthews Hollywood Century 40″ S Stand For Grip Arm Kit, Adorama, Inc, New York, N.Y.). This reusable device was designed without any irregular surfaces to facilitate cleaning with antimicrobial wipes (either Virex or Sani-Cloth) to disinfect it for reuse within following 3 minutes of air-drying. The three openings in the NPFS were smoothed and sealed to ensure that these areas would not harbor unwanted particulate matter. These openings included two separate ¼-inch access ports in the lower midline of the face shield and a 5/16-in. suction port on the midlateral surface.

Prototype evaluation included initial testing with a standard wall suction (Vacutron Suction Regulators by Chemetron, Inc) with a maximum regulated suction of 320±20 mmHg. Additional testing was done with a portable suction (Neptune 3 Waste Management System 120 VAC Rover, Stryker, Kalamazoo, Mich.) which includes a high efficiency particulate air (HEPA) Filter and is capable of generating continuous suction in a range from 50 to 520 mmHg.

All patient-related experiments were performed in a standard examination room designated as having six air changes per hour (ACH) and employed standard wall suction as designated above. Sterile disposable suction tubing (0.6 mm×3.7 mm, nonconductive suction tubing, 12 ft length; Cardinal Health, Waukegan, Ill.) was attached to the NPFS and used through the entire test.

The NPFS wass positioned on a stand and employs a disposable antimicrobial wrap to secure an enclosure around the head. A sterilization wrap (Halyard H100 sterilization wrap, O&M Halyard, Inc, Alpharetta, Ga.) was fashioned as a cylinder and secured to the NPFS with tape circumferentially. The wrap was adapted to drape over the patient's head as a hood to create a closed environment by drawing the lower aspect of the drape loosely around the patient with a 3 to 4 ft length of umbilical tie (white twill ½ inch, 36-yard roll, Horn Textile, Inc, Titusville, Pa.).

The H100 sterilization wrap is a “a disposable infection control product” made of water-repellant breathable “low lint spunbond meltblown spunbond (SMS) fabric” as is also used in production of face masks and lab jackets.

An electronic micromanometer (AirdataMultimeter ADM-870C, Shortridge Instruments, Inc, Scottsdale, Ariz.) was used to measure air pressure in the NPFS during use. Evaluation of the prototype involved assessment of the vacuum generated within the face shield. The probe was placed through one of the anterior ports with H100 sterilization wrap in place around the shield. Pressure readings were obtained both with the second anterior port opened and closed with tape.

Initial testing in human volunteers included assessment of fluctuations in the negative pressure generated within the shield during inspiration and ventilation. Additional pressure testing was done employing a single vs double layer of the sterilization wrap as well as comparison of use of the wall suction on maximum vs portable suction with a HEPA filter.

The prepared NPFS with sterilization wrap applied is rotated into position with the patient leaning slightly forward and adjusting the exam chair to a level optimal for the exam. The most optimal of the two anterior access ports is chosen for use during the procedure. The unused port is sealed with tape. A twill tape is drawn around the sterilization wrap and the patient to improve the seal and optimize negative pressure within the device.

This assembly was successfully used to complete transnasal laryngoscopy in all 30 patients studied. Thirty-one consecutive patients required transnasal laryngoscopy by the senior author (HTH) over a 24-day period beginning May 5, 2020. Limited COVID testing at the facility during this period permitted viral assessment of the single patient with a tracheostomy. This patient was excluded from study, yielding 30 patients who were offered inclusion in the study with all consenting to participate.

Before performing transnasal laryngoscopy, all but one (who deferred anesthesia) of the 30 patients received 1 cc of a mixture of 4% lidocaine with 1% phenylephrine delivered to the nostril through an anterior access port by syringe spray employing the MADgic Laryngo-tracheal mucosal atomization device (Teleflex Medical, Inc, Morrisville, N.C.).

The flexible transnasal laryngoscopies were performed with either a 2.6 mm Olympus ENF-V3 Video or 3.9 mm ENF-VH Olympus Rhinolaryngoscope during all patient encounters.

To further decrease the risk of aerosol spread, a tighter seal may be formed around the laryngoscope access port. In certain cases, clear tape is placed over the access port, through which a narrow slit is created. The laryngoscope is then placed through the taped port, forming a tight barrier around the scope.

Early experience with the implementation of the NPFS provided sufficient confidence in the capacity to control aerosol generation that after the first three patients use of N95's and surgical gowns were discontinued.

Pulse oximetry (Mallinckrodt N-20E Handheld Pulse Oximeter, Nellcor Puritan Bennett, Inc, Pleasanton, Calif.) was performed on each patient prior to placement of the shield, during the procedure with the shield and drape in place and following completion of the procedure.

Patient response to a 4-question survey was performed at the conclusion of the procedure. Patients were asked to rate their tolerance of three factors: claustrophobia, shortness of breath, and pain. Responses were obtained on a 5-point Likert scale, with ratings defined as (1) none, (2) slight, (3) moderate, (4) severe, and (5) intolerable. The fourth question was an open-ended request for feedback on the experience, which was transcribed into the record and read-back for patient approval.

Prototype testing—Measurements at the anterior opening of the NPFS with a single layer of the grade H100 sterilization wrap identified a mean pressure of −0.013 in. of water, obtained from 18 samples at 10 second intervals. This finding was reproduced without change independent of whether the second access port was open or closed with tape.

Pressure measurements with the model in place were obtained at rest, during inspiration, and during expiration. Further measurements were obtained during sequential modifications to the size and shape of the shield as well as methods to occlude the region around the patient's face (combinations of towels, blankets, and positioning). Depending on the cycle of respiration, employing a single barrier wrap and wall suction at 300 mmHg, the pressure varied between −0.002 (expiration) to −0.020 (inspiration) inches of water. Consistently greater vacuum was achieved with a double layer of the barrier wrap and higher suction achievable (530 mmHg) with the Neptune portable suction system.

Duration of procedure. The duration of the procedure was measured from the initial placement of shield and barrier wrap around the patient to its removal and included the nasal spray, a purposeful delay to allow time for the topical application to work, and then performing the laryngoscopy. This duration ranged from 2 minutes and 10 seconds to 6 minutes and 5 seconds. The longest procedure included interaction with our speech pathologist involved to elicit additional vocalization maneuvers with the flexible laryngoscope in place.

Pulse oximetry. Intermittent pulse oximetry monitored the subjects' oxygenation status with frequent inquiries into the comfort of the process. A Friedman test of repeated measures, used due to the nonparametric nature of the data, was performed on the sample of 30 patients to determine if differences in SpO2 were due to the use of the NPFS. Results demonstrated that no statistically significant differences in mean SpO2 through the intervention (Fr=0.17, P=0.92). Specifically, no differences were noted in the SpO2 recorded preprocedure (98±1.3%), intraprocedure (98±1.2%), and postprocedure (98±1.3%) by Dunn's multiple comparisons test (P>0.99) (FIG. 20). One patient with compromised pulmonary function at baseline was maintained on continuous oxygen treatment (3 L of oxygen per minute, administered by nasal cannula) throughout the process and maintained an SpO2 of 100% through the procedure. Tolerance of the design was excellent, with postprocedure questionnaire identifying no shortness of breath (27/30), no claustrophobia (27/30), no pain (29/30), and no significant changes in pulse oximetry.

Patient tolerance. Assessment of tolerance on a 5-point Likert scale was assessed after the procedure. Ratings included (1) none, (2) slight, (3) moderate, (4) severe, and (5) intolerable.

Among the 30 patients, 27 reported no shortness of breath during the procedure, with the remainder only reporting “slight” shortness of breath. Twenty-nine reported no pain in the process. The one patient reporting “slight” pain during the procedure attributed the discomfort to manipulation of the flexible laryngoscope and not the face shield. Twenty-seven patients reported no claustrophobia, with the remainder reporting only “slight” claustrophobia. Among the comments from the three patients who reported “slight” shortness of breath was “shortness of breath wasn't a big deal—just a little nervous.”

Evaluation of scores in each category were significantly less than the compared, arbitrarily agreed-upon, acceptable tolerance score of 2, as determined by the one sample sign test (P<0.0001) (FIG. 21).

Diagnostic laryngoscopy was successfully performed in a negative pressure microenvironment created to limit dispersion of aerosols. Further application of the NPFS device is targeted for use with transnasal laryngeal laser and biopsy procedures to be followed by additional modification to enable intranasal and intraoral procedures in a similar protected environment.

Summary of patient observations. Airflow made it comfortable—better than wearing mask he came in with. Did not like the spray (taste); caused sneeze, cough with face shield in place and [suction] activated. Mentioned the scope in nose was irritating but process not painful.

Surgeon observations. Assembly of instrumentation and preparation of the face shield by assistants before the patient enters the room is facilitated by use of a checklist. It is helpful to optimize patient positioning prior to starting the procedure to minimize need for repositioning during the procedure. Positioning is initiated by adjusting the height of the exam chair, the angle of the stand, and the posture of the patient with the face shield in front of the patient before placing the antimicrobial wrap over the head. The full suction as applied to the NPFS is maintained through the end of the procedure to perform a vacuuming maneuver as the assembly is rotated from the patients face with the blue draping material collapsing into the face shield under suction.

A high level of patient tolerance was identified in this effort to limit dispersion of bioaerosols created during transnasal laryngoscopy. The NPFS has a minimalist design with smooth surfaces that permit rapid disinfection employing antimicrobial wipes followed by air-drying in a 3-minute period before reuse. The NPFS is deployed in a manner that permits it to be immediately rotated away from the patient, if an airway were to become compromised. The NPFS provides an adaptation designed to decrease dissemination of virus and has been successfully used clinically to examine the larynx.

### Example 3

Objective: To evaluate a negative pressure microenvironment designed to contain laser plume during flexible transnasal laryngoscopy.

### Materials and Methods

The Negative Pressure Face Shield (NPFS) which was assessed as well tolerated through initial use on 30 patients, see Examples 1 and 2 above, was used during diagnostic transnasal laryngoscopy on additional 108 consecutive patients evaluated through questionnaires and sequential pulse oximetry. Subsequent study addressed operative transnasal KTP laser laryngoscopy with biopsy done on four patients employing the NPFS.

### Results

As discussed above, the NPFS—a transparent acrylic barrier with two anterior instrumentation ports—can have a variety of configurations, such as that described and used in Examples 1 and 2 above (version 3 or v.3) and configurations including positioning the side suction port close to the level of the nose and having deepened lateral sides with squaring off the lower projection (version 4 or v.4).

A post-procedure questionnaire employing a 5-point Lykert scale ranging from no symptoms (rating of 1) to intolerable (rating of 5) identified patient tolerance among 22 patients evaluated to be excellent and similar in the comparison to the 116 patients. Among the 138 patients analyzed, only one with ‘moderate claustrophobia’ rated the experience as greater than ‘mild’ with 100% of patients reporting either no symptoms or mild pain or shortness of breath. The NPFS was then successfully used for laser laryngoscopy (with biopsy) for patients with laryngeal papilloma and hemorrhagic polyp with post-procedure questionnaire identifying no shortness of breath (4/4), no claustrophobia (4/4), no pain (4/4) and no significant changes in pulse oximetry during use.

### CONCLUSION

Extensive experience in performing diagnostic laryngoscopy with the NPFS directed design changes leading to successful use for transnasal flexible laser laryngoscopy with biopsy in a negative pressure microenvironment.

### Materials and Methods

The NPFS version 3 (v.3) in this study was made of 0.2″ (5 mm) thick acrylic. This NPFS is a transparent 9″×10.5″ rectangular device with a depth of 3.5″ and an inferior stabilization flange used to engage a clamp on a stand for positioning in front of the patient. The three openings in the NPFS were smoothed and sealed to ensure that these areas would not harbor unwanted particulate matter. These openings included two separate ¼-inch (6.35 mm) access ports in the lower midline of the face shield and a 5/16-inch (7.94 mm) suction port on the mid-lateral surface.

A second version of the NPFS was also used in this study—NPFS version 4 or v4—this version included modifications of placing the suction close to the nose and mouth. Also, the sides of the NPFS were deepened to provide to not only increased distance from the patient to the front of the shield, but also to assess use of the lower flange of the box as a chin rest.

All clinical (diagnostic) transnasal laryngoscopies employing the NPFS were done employing standard wall suction (Vacutron® Suction Regulators by Chemetron Inc) with a maximum regulated suction of 320±20 mm Hg.

All operative transnasal laryngoscopies with laser and biopsy were performed employing a portable suction (Neptune® 3 Waste Management System 120 VAC Rover, Stryker, Kalamazoo, Mich.) which includes a High Efficiency Particulate Air (HEPA) Filter at continuous suction approximating 520 mm Hg. Both suction systems were in accordance with recommendations from the FDA for use with a negative pressure “Airway Dome”.

All diagnostic laryngoscopies were performed in a standard examination room designated as having 6 air changes per hour (ACH) and employed standard wall suction as designated above. Sterile disposable suction tubing (0.6 mm×3.7 mm, non-conductive suction tubing, 12 ft length; Cardinal Health® Waukegan Ill.) was attached to the NPFS and used through the entire test.

All operative laser laryngoscopies were done in an operating room with 25 to 30 air changes per hour.

The 109 consecutive patients who were offered diagnostic trans-nasal laryngoscopy with the NPFS by the senior author (HTH) between May 28th to Aug. 6, 2020 are analyzed in this report and exclude one who had no laryngeal symptoms and deferred the examination leaving 138 patients for evaluation.

As discussed herein, a sterilization wrap (Halyard® H100 sterilization wrap, O&M Halyard Inc., Alpharetta, Ga.) was fashioned as a cylinder and secured to the NPFS with tape circumferentially. The wrap was adapted to drape over the patient's head as a hood to create a closed environment by drawing the lower aspect of the drape loosely around the patient either by using a 3-4 foot length of umbilical tie (white twill ½ inch, 36-yard roll, Horn Textile Inc., Titusville, Pa.) or, as was more common practice later in the study, bunching up the drape to tuck into the patient's shirt without use of twill tape.

Initially the topical nasal anesthesia (1 cc of a mixture of 4% lidocaine with 1% phenylephrine) was consistently administered through an anterior access port of the NPFS employing the MADgic Laryngo-tracheal mucosal atomization device (Teleflex Medical, Inc, Morrisville, N.C.). A recent report identified that administration of medical aerosols from nebulizers do not produce a bioaerosol unless they stimulate a cough. Experience consistent with this finding and led to use of standard shorter intranasal mucosal atomization device (MAD Nasal™ Teleflex Medical, Inc, Morrisille, N.C.) with direct spraying of the nose within a several second time frame of removing the mask and placing the NPFS about the patient. For those diagnostic laryngoscopies requiring more extensive topical anesthesia—including spray to the oral cavity during deep inhalation to topically anesthetize the larynx for view of the subglottis—the technique was a previously described through an anterior access port by syringe spray employing the MADgic Laryngo-tracheal mucosal atomization device.

The diagnostic flexible trans-nasal laryngoscopies were performed with either a 2.6 mm Olympus ENF-V3 Video or 3.9 mm ENF-VH Olympus Rhinolaryngoscope during all patient encounters.

The transasnal laser and biopsy procedures were done with a flexible bronchoscope with a 4.2 mm outer diameter and a 2.0 mm working channel (BF-P190 slim bronchoscope Olympus America 3500 Corporate Parkway Center Valley, Pa. 18034-0610). Biopsies were done with 1150 mm long disposable flexible biopsy forceps designed for use through a working channel no smaller than 2.0 mm (Endojaw Disposable Forceps Model No FB-231 D Olympus Medical Systems Corp. Tokyo, Japan).

In all 4 cases of laser laryngoscopy the patients were premedicated with a drying agent (glycopyrrolate 1 mg po verify). Bilateral superior laryngeal nerve blocks were performed 30 to 45 minutes before the procedure in each case and was supplemented by topical nasal decongetion and anesthesia with 4% lidocaine with 1% phenylephrine spray. Final topical anesthesia to the larynx was performed with delivery of ˜2 cc of 4% lidocaine directly to the larynx through a 25 gauge sclerotherapy needle in the channel of the laryngoscope.

Mild sedation (1-2 mg of IV versed) was administered in two of the four cases as they had requested pre-op and was deemed unnecessary by the one of the patients after experiencing the procedure.

The transnasal laser laryngoscopy was done with KTP laser settings of 30 watts, 15 millisecond pulses and 2 pulses per second.

For diagnostic laryngoscopy in clinic, pulse oximetry (Mallinckrodt N-20E Handheld Pulse Oximeter, Nellcor Puritan Bennett Inc, Pleasanton, Calif.) was performed on each patient prior to placement of the shield, during the procedure with the shield and drape in place and following completion of the procedure. Monitoring of continuous pulse oximetry by anesthesia permitted review of recorded oxygen saturations pre, intra- and post procedure.

All patients responded to a 4-question survey performed at the conclusion of the procedure. Patients were asked to rate their tolerance of three factors: claustrophobia, shortness of breath, and pain. Responses were obtained on a 5-point Likert scale, with ratings defined as (1) none, (2) slight, (3) moderate, (4) severe, and (5) intolerable. The fourth question was an open-ended request for feedback on the experience, which was transcribed into the record and read-back for patient approval.

Overall tolerability was analyzed by dichotomizing the Likert scale such that values of (1) none or (2) slight were considered to be ‘well tolerated’ and other values (3, 4, 5) were considered not to be “well tolerated”. This cutoff was determined a priori to define acceptable tolerance. The Clopper-Pearson procedure was used to construct confidence intervals for the true tolerability rate on each of the three factors. To compare tolerability between the two devices configuration of the NPFS a stricter tolerability threshold was applied such that only values of (1) were considered well tolerated, as preliminary data showed all subjects meeting the a priori threshold. Fisher's exact test was used to determine if there were significant differences in the stricter tolerability proportion between groups.

### Results

Oxygen Saturation. Table 1 summarizes the descriptive statistics of oxygen saturation by NPFS version, with the Version 3 group split by inclusion in the preliminary results and the distribution of values for the three groups is shown in FIG. 22. Summaries for Version 4 remain unchanged. Model results show there is not a significant interaction between the two prototypes and time (F(4, 269)=0.99, p=0.4129), and there is also no significant main effect of prototype version (F(2, 135)=1.33, p=0.2671) or time (F(2, 269)=0.38, p=0.6829). This indicates that overall, oxygen saturation did not differ significantly between the two groups on Version 3 and Version 4 or when measured pre-, intra-, or post-procedure. As there was not a significant interaction between version and time, differences between time points can be compared by averaging over the three groups. There were not any significant differences in oxygen saturation between any of the three time points (Table 2).

### Patient Tolerance

Tolerance was assessed on a 5-point Likert scale after the procedure. Ratings included (1) none, (2) slight, (3) moderate, (4) severe, (5) intolerable, as would be readily understood.

Table 3 gives the observed tolerability and 95% confidence intervals for the proportion of subjects that would report the procedure as tolerable on each of the three factors for the subgroups of subjects on Version 3, also shown in FIGS. 23 and 24. In the preliminary group, 100% of the subjects reported meeting the tolerability threshold on all three factors and we have high confidence that the true tolerability rate is above 88%. In the subsequent subjects evaluated on Version 3, there was one subject reporting less than ‘well tolerated’ claustrophobia (considered moderate claustrophobia—not severe or intolerable), but there is still have high confidence that the true tolerability rate for claustrophobia is above 93%. For shortness of breath and pain, all subjects reported these factors as well tolerated and we have high confidence that the true well tolerated rate is above 95% on these two factors. In comparing the stricter tolerability threshold (none vs. any intolerance) between the three groups, the Fisher's exact p-values were 0.4094, 0.2102, and 0.5484 for claustrophobia, shortness of breath, and pain, respectively. These results indicate no significant differences in the strict interpretation of ‘well tolerated’ between the three groups.

The NPFS (v.4) was then successfully used for laser laryngoscopy (with biopsy) for patients with laryngeal papilloma (3) and hemorrhagic polyp (1) with post-procedure questionnaire identifying no shortness of breath (4/4), no claustrophobia (4/4), no pain (4/4) and no significant changes in pulse oximetry during use.

Surgeon Observations. The additional depth provided to the NPFS v.4 permitted use of the lower shelf to serve as a place for the patient to deposit the facial tissue given to them at the end of the case when they could blow their nose and clean their face in a closed environment. The capacity to keep the nose and mouth more than 4 inches from the anterior wall with the suction interspersed prevented any moisture developing on the anterior pane.

Although the disclosure has been described with reference to preferred embodiments, persons skilled in the art will recognize that changes may be made in form and detail without departing from the spirit and scope of the disclosed apparatus, systems and methods.

What is claimed is:

1. A negative pressure face shield comprising:
a. a housing defining a lumen;
b. at least one anterior access port defined within the housing; and
c. a suction port defined within the housing.

2. The negative pressure face shield of claim 1, further comprising flange at an inferior portion of the housing, the flange configured for attachment to a stand.

3. The negative pressure face shield of claim 1, further comprising a sealant disposed around the at least one anterior access port.

4. The negative pressure face shield of claim 3, wherein the sealant comprises one or more of tape, adhesive, gel, oil, wax, epoxy, or jelly.

5. The negative pressure face shield of claim 1, wherein the housing has a depth of at least about 3.5 inches.

6. The negative pressure face shield of claim 1, wherein the anterior access port is disposed at a lower midline of the housing.

7. The negative pressure face shield of claim 1, wherein the suction port is disposed on a side of the housing.

8. A face shield comprising:
(a) a housing comprising:
(i) a face;
(ii) a superior portion;
(iii) an inferior portion; and
(iv) at least first side portion and a second side portion;
(b) at least one access port defined within the face;
(c) at least one suction port defined within the first side portion; and
(d) a hood engaged with at least the superior portion of the housing,
wherein the at least one suction port is configured to be engaged with a filtration system, and
wherein the housing and hood define a lumen where a hermetic environment can be created.

9. The face shield of claim 8, further comprising at least one flange disposed on the inferior portion.

10. The face shield of claim 8, further comprising a sealant disposed around the at least one access port to prevent fluid or pressure transfer across the face via the access port.

11. The face shield of claim 10, wherein the sealant is removable such that the access port may be used for insertion of tools into the hermetic environment.

12. The face shield of claim 8, wherein the hood comprises a fabric.

13. The face shield of claim 8, wherein the housing is sterilizable.

14. The face shield of claim 8, wherein the housing is acrylic.

15. A system for minimizing aerosol spread comprising:
(a) a negative pressure face shield comprising:
(i) a housing defining a lumen;
(ii) at least one anterior access port defined within the housing;
(iii) at least one suction port defined within the housing; and
(iv) a hood engaged with the housing for encircling a head of a patient; and
(b) a filtration system in communication with the at least one suction port via tubing,
wherein a negative pressure environment is created within the lumen.

16. The system of claim 15, further comprising a tie for securing the hood to the patient.

17. The system of claim 15, wherein one or more tools may be inserted into the negative pressure environment via the at least one anterior access port.

18. The system of claim 15, wherein the hood is engaged with the housing via one or more of a strap, Velcro, adhesive, glue, grommets, buttons, tape, and male/female connectors.

19. The system of claim 15, wherein the hood is comprised of a surgical fabric.

20. The system of claim 15, wherein the housing is sterilizable.
